(19) United States
(12) Patent Application Publication
(10) Pub. No.: US 2017/0131250 A1
(43) Pub. Date: May 11, 2017

(54) DEVICE AND METHOD FOR DETECTING A GAS COMPONENT

(71) Applicant: Robert Bosch GmbH, Stuttgart (DE)

(72) Inventor: Tobias Lang, Stuttgart (DE)

(21) Appl. No.: 15/348,368

(22) Filed: Nov. 10, 2016

(30) Foreign Application Priority Data
Nov. 10, 2015 (DE) 10 2015 222 064.8

(51) Int. Cl. G01N 33/00; G01N 25/18

(52) U.S. Cl. CPC G01N 33/005; G01N 25/18; G01N 33/0062

(57) ABSTRACT

A device and a method are described for detecting a gas component. The device is designed with: a detection device for detecting a time-dependent raw signal which indicates a gas concentration of the gas component to be detected; a provision device for providing at least one time-dependent additional measured variable which indicates a cross-influence on the detection of the raw signal; a compensation device for generating an intermediate signal based on the raw signal and the at least one detected additional measured variable; and a computing device which is designed for processing the raw signal based on temporally preceding values of the detected raw signal and/or temporally preceding values of the at least one detected additional measured variable in order to generate an output signal which indicates the gas concentration of the gas component to be detected.

### FIELD OF THE INVENTION

The present invention relates to a device and a method for detecting a gas component, in particular for cross-influence-compensated detection of a gas component, preferably hydrogen.

### BACKGROUND INFORMATION

Devices for detecting gases, so-called gas sensors, are required for numerous applications. One technique that may be used is based on heat conductivity measurements. Heat is released at a first area of the gas sensor, generally a diaphragm, and a temperature is measured at a second area of the gas sensor. A gas to be detected is conducted in such a way that a heat conductivity of the gas sensor between the first and the second area changes as a function of the gas. The gas may be detected, based on the heat released at the first area and the measured temperature, and generally based on a temperature of the diaphragm. Uncompensated cross-influences, for example due to air humidity or various ambient temperatures around the gas sensor, may distort the detection result.

A gas sensor is described in German Published Patent Application No. 42 44 224 A1, with the aid of which a concentration or a change in the concentration of gaseous foreign substances in gases is determined according to the heat conductivity principle.

### SUMMARY

The present invention provides a device as described below.

Accordingly, a device for detecting a gas component is provided which includes: a detection device for detecting a time-dependent raw signal which indicates a gas concentration of the gas component to be detected; a provision device for providing at least one time-dependent additional measured variable which indicates a cross-influence on the detection of the raw signal; a compensation device for generating an intermediate signal based on the raw signal and the at least one detected additional measured variable; and a computing device which is designed for processing, in particular correcting, the raw signal based on temporally preceding values of the detected raw signal and/or of the at least one detected additional measured variable, in particular temporally preceding values of the intermediate signal, in order to generate an output signal which indicates the gas concentration of the gas component to be detected.

“Temporally preceding values” are understood to mean values that have been detected for the same particular variable, but at an earlier point in time. A memory device may be provided in the device, for example as part of the computing device, for buffering the detected values.

In addition, a method for detecting a gas is provided, including the following steps: detecting a time-dependent raw signal which indicates a gas concentration of the gas component to be detected; providing at least one time-dependent additional measured variable which indicates a cross-influence on the detection of the raw signal; generating an intermediate signal based on the raw signal and the at least one detected additional measured variable; and generating an output signal which indicates the gas concentration of the gas component to be detected, by processing, in particular correcting, the raw signal based on temporally preceding values of the detected raw signal and/or temporally preceding values of the at least one detected additional measured variable, in particular temporally preceding values of the intermediate signal.

### Advantages of the Invention

The concept underlying the present invention lies in reducing or entirely eliminating interfering cross-influences or cross-sensitivities in the detection of a gas component in a particularly simple and technically easily implementable manner. The device according to the present invention, which is designed, for example, as a gas concentration sensor based on heat conductivity measurements, is thus particularly insensitive to changing environmental conditions, for example temperature, other mixture components such as oxygen or air humidity, etc., as cross-influences. The detection of the gas component may in particular include or involve the detection of the gas concentration of the gas component in a fluid or gas mixture, the fluid or gas mixture also including further gas components.

The particular cross-influence by these environmental conditions, which are detectable as additional measured variables, is advantageously reducible or eliminatable according to the present invention without the corresponding additional measured variable having to be known with great accuracy. Cross-sensitivities which are slow-acting and relatively imprecise, or known only with large variations, are advantageously compensated for without slowing down a response to the gas component to be detected. Balancing between signal noise or measuring accuracy on the one hand and a response time on the other hand, which is often necessary, may thus be avoided or eliminated.

The device according to the present invention for detecting a gas component, for example for detecting a hydrogen concentration in a hydrogen-air mixture, is usable, for example, in a hydrogen safety sensor for automotive fuel cell systems or for moisture measurement in internal combustion engines, in particular when the device is installed downstream from a turbocharger. Other possible fields of application include fuel cell systems for energy generation, for example for engine applications or separately from engine applications, hydrogen internal combustion engines, safety sensors in facilities for hydrogen generation (for example, electrolysis with the aid of regeneratively produced power), general gas analytical tasks (gas chromatography. for example), and others.

According to one preferred refinement, for providing one of the at least one additional measured variables, the provision device includes an additional sensor which is designed for measuring the additional measured variable in question. The additional sensor may be an additional temperature sensor, for example, which is designed for measuring an ambient temperature. The additional sensor may be situated, for example, on a substrate of the device.

According to another preferred refinement, the detection device is designed for carrying out a heat conductivity measurement for detecting the raw signal, a heating resistor being designed for heating a diaphragm in a substrate, a temperature sensing device being designed for measuring a temperature at an area of the substrate situated at a distance from the diaphragm, and the detection of the raw signal being based on the heating power of the heating resistor and based on a temperature difference between a temperature of the heating resistor and the temperature detected by the temperature sensing device. A heating resistor is understood to mean an ohmic resistor which is situated in particular on the diaphragm.

According to another preferred refinement, for detecting at least one of the at least one additional measured variables, the provision device includes an interface, with the aid of which the additional measured variable in question is receivable from an external measuring device. The device according to the present invention may thus be used in a particularly versatile way, in that it is connectable to various external measuring devices, depending on the planned site of operation. The provision device may also include at least one additional sensor and also at least one interface, as described above.

Additionally or alternatively, the provision device may be designed for controlling the detection device, which is designed for detecting the time-dependent raw signal, in such a way that the detection device itself is also operated for detecting the at least one additional measured variable. If the detection device is designed, for example, for carrying out a heat conductivity measurement as described above, for providing the at least one time-dependent additional measured variable, the provision device may be designed for carrying out the heat conductivity measurement at a number p of different heater temperatures, i.e., p different temperatures of the heating resistor. In this way, a new heat conduction equation may be established for each selected temperature, and thus also solved according to the same number of variables. In addition to the raw signal as a summary variable, further measured values may thus be generated which may be bijectively mapped onto the p-tuple of gas concentrations c_{gas}, c_{gas2}, . . . of all gas components involved. In this way, for example, an air humidity content which is provided by the provision device as an additional measured variable may be determined directly from the heat conductivity measurements without a separate air humidity measurement.

According to another preferred refinement, the gas component to be detected is hydrogen; i.e., a hydrogen concentration is detected. According to another preferred refinement, at least one of the additional measured variables is air humidity, i.e., the air humidity in a gas mixture, of which the gas component to be detected, for example hydrogen, represents one among multiple gas components.

According to one preferred refinement of the method according to the present invention, for detecting the raw signal a heat conductivity measurement is carried out, a heating resistor being used to heat a diaphragm in a substrate, a temperature sensing device being used to measure a temperature at an area of the substrate situated at a distance from the diaphragm, and the detection of the raw signal being based on the heating power of the heating resistor and based on a temperature difference between a temperature of the heating resistor and the temperature detected by the temperature sensing device.

According to another preferred refinement, for providing the at least one additional measured variable a number of heat conductivity measurements are carried out at different heater temperatures, i.e., temperatures of the heating resistor for heating the diaphragm in the substrate.

### BRIEF DESCRIPTION OF THE DRAWINGS

FIG. 1 shows a schematic block diagram of a device for detecting a gas component according to one specific embodiment of the present invention.

FIGS. 2 through 4 show schematic graphs for explaining a mode of operation of the device from FIG. 1.

FIG. 5 shows a schematic flow chart for explaining a method for detecting a gas component according to another specific embodiment of the present invention; and

FIG. 6 shows a schematic cross-sectional view of a micromechanical heat conductivity sensor which is usable as a detection device for the device from FIG. 1.

### DETAILED DESCRIPTION

Unless stated otherwise, identical or functionally equivalent elements and devices are provided with the same reference numerals. The numbering of method steps is used for clarity, and unless stated otherwise, in particular is not intended to imply a specific chronological sequence. In particular, multiple method steps may also be carried out simultaneously.

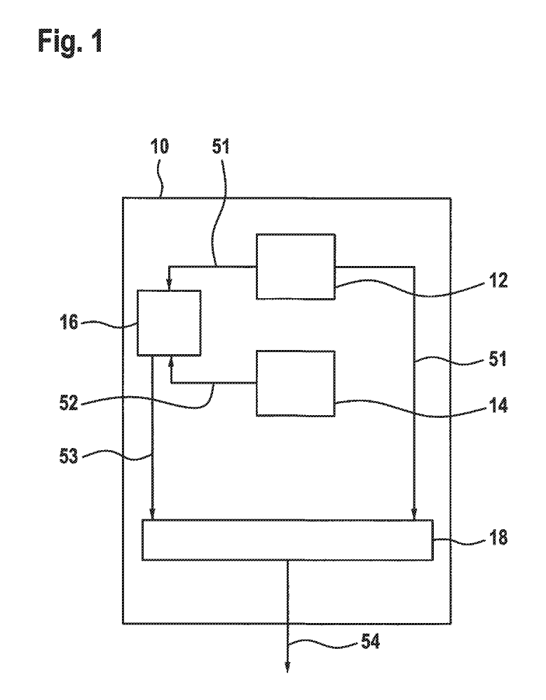

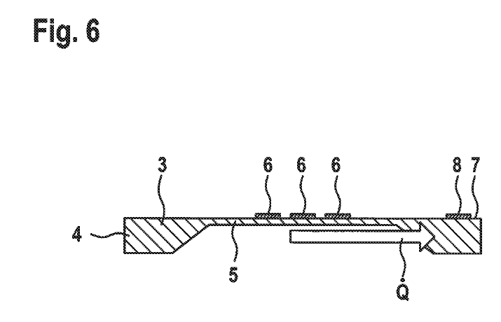

FIG. 1 shows a schematic block diagram of a device 10 for detecting a gas component according to one specific embodiment of the present invention. Device 10 includes a detection device 12 for detecting S01 a time-dependent raw signal 51 which indicates the gas component to be detected. Device 10 is described below by way of example using a micromechanical heat conductivity sensor as detection device 12, which is designed for detecting a hydrogen concentration in a gas mixture, based on the heat conductivity measurement. It is to be understood that alternatively or additionally, device 10 may include a plurality of other detection devices which are based on other measuring techniques and/or which are designed for detecting other gas components. Reference is made to FIG. 6 for explaining the functioning of the heat conductivity sensor as detection device 12.

FIG. 6 shows a schematic cross-sectional view of a micromechanical heat conductivity sensor 3 which is usable as detection device 12 for device 10 according to the present invention. Heat conductivity sensor 3 includes a substrate 4 in which a diaphragm 5 is provided. At least one heating resistor 6, situated on diaphragm 5, is designed for heating a measuring gas layer which adjoins diaphragm 5, i.e., a layer of the gas mixture in which the gas component to be detected is to be detected, to a predetermined temperature Tₕₑₐₜₑᵣ, which may also be referred to as the heater temperature, with a known heating power P. Temperature Tₕₑₐₜₑᵣ may also be changeable, for example by the provision device. Diaphragm 5 is designed as a thin silicon oxide diaphragm in order for a total heat flow \dot{Q} which is to be ascertained to be influenced preferably strongly by a gas composition of the gas mixture in comparison to a more or less constant heat conduction through solid structures of heat conductivity sensor 3 which is to be kept preferably low.

After diaphragm 5 is heated, a total heat flow \dot{Q} from heating resistor 6 to a cooler external area 7 of heat conductivity sensor 3, made of silicon, for example, results, whose temperature T_{chip} is ascertainable by a temperature sensing device 8 situated in area 7, and which is thus known. Total heat flow \dot{Q} is identical to heating power P, and is made up of various components. Disregarding convective components and radiation components, the total heat flow results from the following heat conduction equation:

\dot{Q}=(Gₘₑₘλₘₑₘ+G_{gas}λ_{gas}(c_{gas1}, c_{gas2}, . . . , T_{gas}) ) (Tₕₑₐₜₑᵣ−T_{chip}),

where Gₘₑₘ and G_{gas} are geometric factors, λₘₑₘ is a heat conductivity of diaphragm 5, and λ_{gas} is a heat conductivity of the gas mixture. Heat conductivity λ_{gas} of the gas mixture, which may also be referred to as the measuring gas, generally is a function not only of the composition of the gas mixture, i.e., gas concentrations c_{gas1}, c_{gas2}, . . . of gas components in the gas mixture, but also of a gas temperature T_{gas} of the gas mixture; i.e., a so-called “temperature coefficient” contributes.

Via at least one electrical connection of heat conductivity sensor 3, an electrical measurement may be carried out which correlates with the heat conductivity of the gas mixture, and thus its composition, i.e., the concentrations of the gas components, and which thus allows a concentration measurement, i.e., detection of the gas component to be detected.

Based on this measurement and optionally further pieces of information internal or external to the sensor, with the aid of an analog and/or digital signal processor, for example, time-dependent raw signal 51 having values X₁(t) may be generated, which represents a measure for the concentration of the gas component to be detected, i.e., indicates a gas concentration of the gas component to be detected. However, raw signal 51 generally is still distorted by cross-influences. Raw signal 51 may be, for example, an analog or digitized bridge voltage of a resistance measuring bridge, or may correspond to such a bridge voltage. Alternatively, raw signal 51 may be a function of heating power P, of heater voltage at heating resistor 6, or of the heater current, the heating resistor being controlled in such a way that a constant temperature difference, Tₕₑₐₜₑᵣ−T_{chip}, results between the heating resistor and area 7 of heat conductivity sensor 3.

As another alternative, the heater voltage or the heater current or some other variable formed therefrom may be predefined, and raw signal 51 may be formed from mentioned temperature difference, Tₕₑₐₜₑᵣ−T_{chip}. Other options, not listed here in greater detail, for forming raw signal 51 as part of the detection of raw signal 51 are possible which ultimately rely on variables contained in the heat conduction equation given above, or on functions of these variables. Thus, for example, a cooling time curve of heating resistor 6 or of a temperature sensor on diaphragm 5 may be ascertained according to a temporally modulated or pulse-shaped heater current and used for forming raw signal 51.

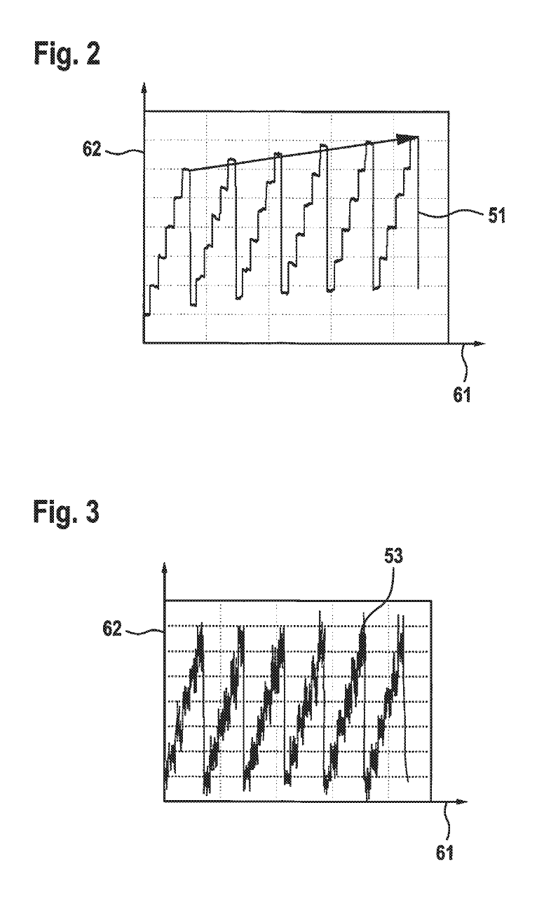

FIG. 2 shows a graph which depicts by way of example a time curve of value X₁(t) of raw signal 51. A vertical axis 62 of the graph in FIG. 2 shows a hydrogen concentration in vol-%, and a horizontal axis 61 shows a point in time t (or equivalently, a number of measurements). In the following, a special case in which only a single additional measured variable 52, namely, the air humidity, is discussed by way of example. In other words, only the air humidity is taken into account as a cross-influence variable, i.e., as a physical variable which has a cross-influence on the detection of raw signal 51.

Raw signal 51 having value X₁(t) may be defined, for example, as a linear function

X₁(t)=a·λ_{gas}+b

of the combined variable

λ_{gas}˜Pₕₑₐₜₑᵣ/(Tₕₑₐₜₑᵣ−T_{chip}),

where the tilde (˜) indicates a direct proportionality. Coefficients a and b of the linear function may be empirically adjusted in such a way that, with dry air and a preset reference temperature, raw signal 51, having value X₁(t) at point in time t, is identical to a hydrogen concentration that is present in a hydrogen-air mixture in question. After such “balancing,” X₁(t), at least at the selected reference temperature and with dry air, corresponds directly to the hydrogen concentration, i.e., the gas concentration of the gas component to be detected.

FIG. 2 illustrates a case by way of example in which the hydrogen concentration has increased step-by-step from 0 vol-% to 10 vol-% in increments of 2 vol-%, and has then been set back to 0 vol-%, the operation thus described being repeated cyclically, in the present example, six times. In addition, in each complete cycle of hydrogen concentrations, a different air humidity component has been set, the air humidity component having been increased from 0 vol-% to 25 vol-%, from left to right in FIG. 2, in increments of 5 vol-%. The signal pattern illustrated in FIG. 2 shows small variations, but shows a cross-influence to be expected due to the higher conductivity of moist air, i.e., air in the hydrogen-air mixture having higher air humidity.

Device 10 also includes a provision device 14 for providing S02 at least one time-dependent additional measured variable 52 which indicates a cross-influence on detection S01 of raw signal 51. As described above, in the present case the focus by way of example is on air humidity as additional measured variable 52.

Device 10 also includes a compensation device 16 for generating an intermediate signal 53 based on raw signal 51 and at least one detected additional measured variable 52. Such a generated intermediate signal 53 having a value X₂(t) is depicted by way of example in FIG. 3. For generating intermediate signal 53, a quickly responding and correspondingly noisy air humidity signal has been provided, for example by an additional sensor of provision device 14, as additional measured variable 52 having time-dependent value Y(t), and raw signal 51 having time-dependent value X₁(t) has been corrected by an appropriate error value. Intermediate signal 53 has time-dependent value X₂(t).

In other words, an intermediate signal 53 having a time-dependent value X₂(t) has been generated from the heat conductivity measurement as function X₂(t)=f(X₁(t), Y(t)) with the aid of the additional measured variable having time-dependent value X₂(t) and raw signal 51, which is distorted by cross-sensitivity, having value X₁(t). When multiple additional measured variables Y₁, Y₂, . . . are provided by provision device 14, X₂(t) is generated as X₂(t)=f(X₁(t), Y₁(t), Y₂(t), . . . )Intermediate signal 53 represents a measure for the gas component to be detected, in particular for a gas concentration of the gas component to be detected, which is corrected for the cross-sensitivity. Intermediate signal 53 may thus also be referred to as a “precompensated signal.” By definition, intermediate signal 53 on a time average is no longer distorted by cross-sensitivity, but may show scattering or noisy characteristics, which may result in a loss of information concerning the change over time of the gas component to be detected.

Function f is preferably selected in such a way that a separation of the gas concentration to be detected and additional measured variable 52 takes place. In a simple case, function f may be an affine mapping, i.e., a coordinate transformation. Value Y(t) of the air humidity as additional measured variable 52 may be ascertained by provision device 14, for example as a change, i.e., increase, in a heat conductivity across temperature Tₕₑₐₜₑᵣ of heating resistor 6.

The design of function f depends on which environmental conditions are mapped by individual additional measured variable(s) 52, and how it/they act in the form of cross-sensitivities. Function f may be implemented by analytical computations or also by empirically parameterized models, for example polynomials or characteristic maps.

FIG. 3 shows an example of another graph having the same axes 61, 62 as FIG. 2, in which values X₂(t) of intermediate signal 53 are plotted as a function of time.

Device 10 also includes a computing device 18 which is designed for correcting raw signal 51, based on temporally preceding values of detected raw signal 51 and/or of the at least one detected additional measured variable 52, in order to generate an output signal 54 which has a time-dependent value X_{corr}(t) and which indicates the gas concentration of the gas component to be detected.

Output signal 54 having time-dependent value X_{corr}(t) is computed in particular as:

X_{corr}(t):=X₁(t)−g[X₁(t₁, . . . tₖ], Y(t₁, . . . tₖ)],

where

$$g [ {X}_{1} ( {t}_{1} , … {t}_{k} ) , Y ( {t}_{1} , … {t}_{k} ) ] = {\sum }_{j = 1}^{k} \frac{[ {X}_{1} ( {t}_{j} ) - {X}_{2} ( {t}_{j} ) ]}{k} .$$

Points in time t₁, . . . , tₖ are understood to mean temporally preceding measuring points in time at which raw signal 51 and/or additional measured variable 52 having value Y(t) or additional measured variables 52 having values Y_{1, . . . n}(t) have/has been determined, where tₖ<tₖ₋₁< . . . <t₁<t. Use of the temporally preceding values is particularly advantageous under the assumption that rapid changes in gas concentration of the gas component to be detected are to be detected at the same time with high accuracy, and values Y and Y_{1, . . . n}of additional measured variable(s) 52 change much more slowly in comparison.

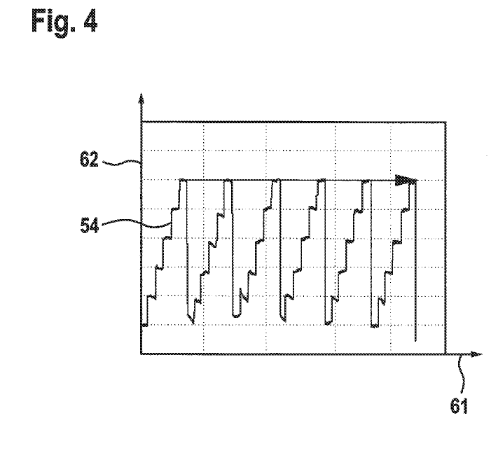

The last term in the equation for function g corresponds to a sliding average value formation of the difference between raw signal 51 and intermediate signal 53. In other words, raw signal 51 is advantageously corrected based on temporally preceding values X₁(t) of raw signal 51 and also based on temporally preceding values X₂(t) of intermediate signal 53. A corresponding resulting output signal 54 having values X_{corr}(t) is illustrated as an example in FIG. 4. Instead of being defined by arithmetic average value formation, function g may be defined in some other way, as long as the scattering and the noise of individual differences X₁(tⱼ)−X₂(tⱼ) are thus reduced. A low pass filter or a sliding median filter, for example, is suitable. The latter is more robust, for example, against individual signal disturbances than simple average value formation, although it results in a slightly higher level of computational effort.

FIG. 4 shows a graph having the same axes as FIG. 2 and FIG. 3. It is clearly apparent in FIG. 4 that the cross-influence from FIG. 2 is compensated for, and also the noise from FIG. 3 is eliminated, without increasing the response time of device 10 according to the present invention.

With a suitable selection of functions f and g, output signal 54 is a particularly low-noise signal which selectively responds quickly and with high accuracy to changes in the gas concentration of the gas component to be measured, without being distorted by the cross-sensitivities. The design of function g with the aid of a sliding average value formation, as described above, is only one option of many.

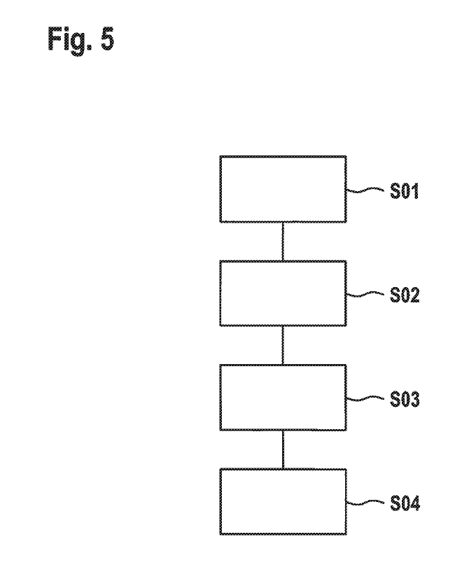

FIG. 5 shows a schematic flow chart for explaining a method for detecting a gas component according to another specific embodiment of the present invention.

The method according to FIG. 5 may advantageously be carried out using the device according to the present invention, in particular device 10, and is adaptable with respect to all variants and refinements described with regard to the device according to the present invention, in particular device 10, and vice versa.

A time-dependent raw signal 51 which indicates a gas concentration of the gas component to be detected is determined in a step S01. At least one time-dependent additional measured variable 52 which indicates a cross-influence on determination S01 of raw signal 51 is provided in a step S02. An intermediate signal 53 based on raw signal 51 and the at least one detected additional measured variable 52 is generated in a step S03. An output signal 54 which indicates the gas concentration of the gas component to be detected, by correcting raw signal 51 based on temporally preceding values of detected raw signal 51 and/or of the at least one detected additional measured variable 52, is generated in a step S04.

The provided method may be integrated into a device for detecting a gas component, in particular when additional measured variable(s) 52 is/are present in the form of variables internal to the sensor, such as heat conductivity measured values which are ascertained under different settings of the heating power of heating resistors. As a result, it may be unnecessary for the device to obtain additional information from the surroundings or from an overall system, for example via a bidirectional interface.

Alternatively, the provided method may be applied externally to the sensor, for example in a system control device, incorporating further information concerning the overall system. An overall system is understood to mean in particular a device for detecting a gas component together with another device which includes or provides the gas component to be detected, such as a fuel cell or an engine, and optionally a system control device.

Alternatively, the method may also be used twice, once internally to the sensor based on variables internal to the sensor as additional measured variables 52, and once externally to the sensor, incorporating further system information concerning the overall system, as additional measured variables 52, which are receivable, for example, by provision device 14 of device 10 according to the present invention via an interface.

What is claimed is:

1. A device for detecting a gas component, comprising:
a detection device for detecting a time-dependent raw signal that indicates a gas concentration of the gas component to be detected;
a provision device for providing at least one time-dependent additional measured variable that indicates a cross-influence on a detection of the raw signal;
a compensation device for generating an intermediate signal based on the raw signal and the at least one detected additional measured variable; and
a computing device for processing the raw signal based on at least one of temporally preceding values of the detected raw signal and temporally preceding values of the at least one detected additional measured variable in order to generate an output signal which indicates the gas concentration of the gas component to be detected.

2. The device as recited in claim 1, wherein to provide one of the at least one additional measured variables, the provision device includes an additional sensor for measuring the additional measured variable in question.

3. The device as recited in claim 1, further comprising:
a heating resistor for heating a diaphragm in a substrate; and
a temperature sensing device for measuring a temperature T_{chip} at an area of the substrate situated at a distance from the diaphragm, wherein:
the detection device performs a heat conductivity measurement for detecting the raw signal, and
a detection of the raw signal by the detection device is based on a heating power P of the heating resistor and on a temperature difference between a temperature Tₕₑₐₜₑᵣ of the heating resistor and on the temperature T_{chip} detected by the temperature sensing device.

4. The device as recited in claim 1, wherein to provide at least one of the at least one additional measured variables, the provision device includes an interface, with the aid of which the additional measured variable in question is receivable from an external measuring device.

5. The device as recited in claim 1, wherein the gas component to be detected is hydrogen.

6. The device as recited in claim 1, wherein one of the at least one additional measured variables is air humidity.

7. A method for detecting a gas component, comprising:
detecting a time-dependent raw signal that indicates a gas concentration of the gas component to be detected;
providing at least one time-dependent additional measured variable that indicates a cross-influence on a detection of the raw signal;
generating an intermediate signal based on the raw signal and the at least one detected additional measured variable; and
generating an output signal that indicates the gas concentration of the gas component to be detected, by processing the raw signal based on at least one of temporally preceding values of the detected raw signal and temporally preceding values of the at least one detected additional measured variable.

8. The method as recited in claim 7, further comprising:
performing a heat conductivity measurement to detect the raw signal;
providing a heating resistor for heating a diaphragm in a substrate;
providing a temperature sensing device for measuring a temperature T_{chip} at an area of the substrate situated at a distance from the diaphragm; and
basing the detection of the raw signal on a heating power of the heating resistor and on a temperature difference between a temperature Tₕₑₐₜₑᵣ of the heating resistor and on the temperature T_{chip} detected by the temperature sensing device.

9. The method as recited in claim 8, wherein the providing of the at least one additional measured variable includes performing a number of heat conductivity measurements at different temperatures Tₕₑₐₜₑᵣ of the heating resistor for heating the diaphragm in the substrate.

10. The method as recited in claim 7, wherein:
the gas component to be detected is hydrogen, and
one of the at least one additional measured variables is air humidity.
